(19) United States
(12) Patent Application Publication
(10) Pub. No.: US 2018/0078404 A1
(43) Pub. Date: Mar. 22, 2018

(54) DEVICE FOR FITTING TO A USER'S MANDIBLE

(71) Applicant: JOHANNES C. BASSON, ENFIELD (GB)

(72) Inventor: JOHANNES C. BASSON, ENFIELD (GB)

(21) Appl. No.: 15/702,282

(22) Filed: Sep. 12, 2017

(30) Foreign Application Priority Data
Sep. 20, 2016 (GB) 1615948.5

(51) Int. Cl. A61F 5/56

(52) U.S. Cl. CPC A61F 5/566

(57) ABSTRACT

A device for fitting to a user's mandible such that the mandible is held in an advanced position. The device comprises: an elongate member comprising a first end portion, a central portion, a second end portion and at least one securement point; and a support member extending from the securement point to, in use, maintain the device in a maintenance position on the user's face; wherein the first end portion is arranged to engage one ramus of the mandible, the second end portion is arranged to engage the other ramus of the mandible, the central portion is arranged to engage a first area of the user's face or head other than the mandible, and the support member is arranged to engage a second area of the user's face or head other than the mandible.

### FIELD OF THE INVENTION

The present invention relates to a device for fitting to a user's mandible, and in particular relates to an anti-snoring or emergency care device.

### BACKGROUND

Studies have shown that approximately 24% of men and 13.8% of women snore habitually, this rising to approximately 60% of men and approximately 40% of women when over the age of 60.

Aside from the strain it can place on relationships, snoring is also associated with various health problems. Interrupted, low-quality sleep caused by snoring leads to drowsiness and fatigue the following day, with resultant loss of concentration and productivity in the workplace. Sleep apnoea is a relatively more serious problem whereby the snorer stops breathing for longer than ten seconds at a time. Sleep apnoea is linked with serious health conditions including stroke, ischaemic heart disease, irregular heartbeat, acid reflux, headaches, mental health issues (such as depression), and more.

Snoring is caused when, during sleep, the muscles in the mouth, tongue and throat, relax. If they relax too much, the jaw and tongue collapse and block the airway, thus restricting the flow of oxygen to the lungs, heart and brain. When breathing, the muscles and uvula vibrate and knock against the back of the throat, causing the well-known snoring sound.

One attempt at addressing snoring is the use of devices developed for preventing the jaw and tongue from collapsing and blocking the airway. These so-called mandibular advancement devices come in various shapes and sizes, but all of them essentially resemble mouth guards that are used in contact sports to prevent fractures to the facial bones. The difference is that mandibular advancement devices generally cover both the upper (maxillae) and lower (mandible) jaws. They are held in position by precise indentations matching the teeth of the wearer.

The medical profession regards the use of mandibular advancement devices as the best known method to lessen or prevent snoring. However, there are several problems associated with these devices, namely:
they are frequently expelled during sleep;
they are retained inside the mouth; and
they cause gum and/or dental pain due to the sustained traction exerted on the teeth.

These problems are more acute in the elderly, due to the fact that their teeth and gums are (usually) not in the best of health. Having missing teeth, as many older people do, can compound the problem, making it difficult or impossible to use a mandibular advancement device.

There therefore remains a need in the art to provide an improved anti-snoring device, and in particular a device that addresses at least some of the above-identified disadvantages of current mandibular advancement devices.

Further, in certain emergency situations, the first step is to ensure that a person's airway is open and stays open. Such a step applies in a large variety of scenarios, and is necessary to ensure a flow of air to the lungs. A common manner of opening an airway is to tilt a person's head back while they are lying down. When the head is back, the airway is typically open (if unobstructed), however maintaining such an airway open requires keeping the head back by minimising movement of the head and monitoring. There is therefore a need in the art to open a person's airway, and maintain the airway open, in a more efficient manner.

### SUMMARY

According to an aspect, a device for fitting to a user's mandible, such that the mandible is held in an advanced position, is provided. The device comprises: an elongate member comprising a first end portion, a central portion, a second end portion and at least one securement point; and a support member extending from the securement point to, in use, maintain the device in a maintenance position on the user's face; wherein the first end portion is arranged to engage one ramus of the mandible, the second end portion is arranged to engage the other ramus of the mandible, the central portion is arranged to engage a first area of the user's face or head other than the mandible, and the support member is arranged to engage a second area of the user's face or head other than the mandible.

By virtue of the support member, the device can be maintained in a maintenance position on the user's face when in use. While in the maintenance position, the engagement of the first and second end portions of the elongate member with the rami of the mandible, and the engagement of the central portion of the elongate member with an area of the user's face or head other than the mandible, advances the mandible into an un-collapsed position. The support member may maintain the device on the user's face by preventing the elongate member from sliding or falling downwardly off the user's face. Various configurations of the support member can achieve this, as described in the various embodiments below. The combination of the elongate member and the support member therefore ensures that the device can be maintained on the user's face, while being able to hold the mandible in an advanced or un-collapsed position. Holding the mandible in such a position not only prevents snoring, but also maintains the user's airway open.

Optionally, the support member comprises a first section attached to a first securement point of the elongate member between the first end portion and the central portion, and a second section attached to a second securement point of the elongate member between the second end portion and the central portion.

Optionally, the first and second sections together form a loop arranged such that, in use, the user's nose protrudes between the elongate member and the support member.

Optionally, the elongate member and support member are integrally formed.

Optionally, the first area comprises the user's maxilla.

Optionally, the first area comprises the user's nasal bone.

Optionally, the second area comprises the user's nasal bone.

Optionally, the second area comprises the user's frontal bone and/or parietal bone.

Optionally, the first section is arranged to engage one of the user's ears, and the second section is arranged to engage another of the user's ears.

Optionally, the first section is arranged to engage the one of the user's ears behind the helix of the ear, and the second section is arranged to engage the other of the user's ears behind the helix of the other ear.

Optionally, at least part of the elongate member comprises a mouldable material.

Optionally, at least part of the support member comprises a mouldable material.

Optionally, the mouldable material is a fibreglass substrate and a polyurethane resin system, or a non-fibreglass polyester.

Optionally, the mouldable material of the elongate member is arranged to be moulded such that the engagement of the first and second end portions with the two mandibular rami, and the engagement of the central portion with the first area, is provided.

Optionally, the mouldable material of the support member is arranged to be moulded such that the engagement with the second portion is provided.

Optionally, the support member, in use, maintains the device in the maintenance position by preventing the elongate member from sliding or falling downwardly away from the top of the user's head.

Optionally, at least one of the first and second sections of the support member, in use, prevents the elongate member from sliding or falling downwardly away from the top of the user's head by engaging with the user's nasal bone, thereby maintaining the device in the maintenance position.

Optionally, at least one of the first and second sections of the support member, in use, prevents the elongate member from sliding or falling downwardly away from the top of the user's head by engaging with the user's frontal bone and/or parietal bone, thereby maintaining the device in the maintenance position.

Optionally, at least one of the first and second sections of the support member, in use, prevents the elongate member from sliding or falling downwardly away from the top of the user's head by engaging at least one of the user's ears, thereby maintaining the device in the maintenance position.

Optionally, at least part of the elongate member comprises an elastic material.

Optionally, at least part of the support member comprises an elastic material.

Optionally, the first and second end portions are further arranged to engage the mandibular angle adjacent to each ramus.

Optionally, the elongate member is arranged such that, when the first and second end portions are engaged with the two mandibular rami, and the central portion is engaged with the first area, a bias force is exerted between the first end portion and the central portion and between the second end portion and the central portion, the bias force being such that the user's mandible is biased into the advanced position, the bias force being in a direction substantially along a line passing from the first or second end portion to the central portion.

Optionally, the support member is arranged such that, when the first and second end portions are engaged with the two mandibular rami, a bias force is exerted along the first and second sections to bias the elongate member in a direction substantially towards the top of the user's head, thereby maintaining the device in the maintenance position.

Optionally, the first and second end portions are mouldable to engage the respective first and second mandibular ramus.

Optionally, at least one of the first end portion, the second end portion, the central portion, and the support member is mouldable to provide the fitting to a user's mandible.

Optionally, at least one of the first end portion, the second end portion, the central portion, and the support member is stretchable to provide the fitting to a user's mandible.

Optionally, the elongate member and/or the support member comprises a malleable material.

Optionally, the malleable material is a metal or metal alloy.

Accordingly to another aspect, a method of fitting the device to a user's mandible such that the mandible is held in an advanced position, is provided. The method comprises: while at least part of the elongate member is mouldable and while the mandible is in the advanced position, placing the elongate member across the user's face such that the first and second end portions extend around the each mandibular ramus, and the central portion is in contact with the first area of the user's face or head; placing the support member over the second area of the user's face or head; bending the first end portion around one ramus to engage the first end portion with the ramus; bending the second end portion around the other ramus to engage the second end portion with the other ramus; waiting a period of time or providing a hardening process, such that the mouldable material of the elongate member becomes rigid.

Further optional features are defined in the dependent claims.

### FIGURE LISTING

Specific embodiments in which the invention is embodied are described below by way of example only and with reference to the accompanying drawings, in which:

FIG. 1 is a known mandibular advancement device.

FIG. 2 is an anatomical drawing of a human skull.

FIG. 3 is a schematic view of a device in accordance with a first embodiment.

FIG. 4a is a side profile view of the device of FIG. 3 when fitted to a user.

FIG. 4b is a side profile view of a device in accordance with a second embodiment, when fitted to a user.

FIG. 5 is a side profile view of a device in accordance with a third embodiment, when fitted to a user.

FIG. 6 is a schematic view of a device in accordance with a fourth embodiment.

FIG. 7 is a side profile view of the device of FIG. 6 when fitted to a user.

### DETAILED DESCRIPTION

The present invention seeks to provide a device for fitting to a user's mandible, and in particular seeks to provide an improved anti-snoring device or an emergency care device.

Whilst various embodiments of the invention are described below, the invention is not limited to these embodiments, and variations of these embodiments may well fall within the scope of the invention which is to be limited only by the appended claims.

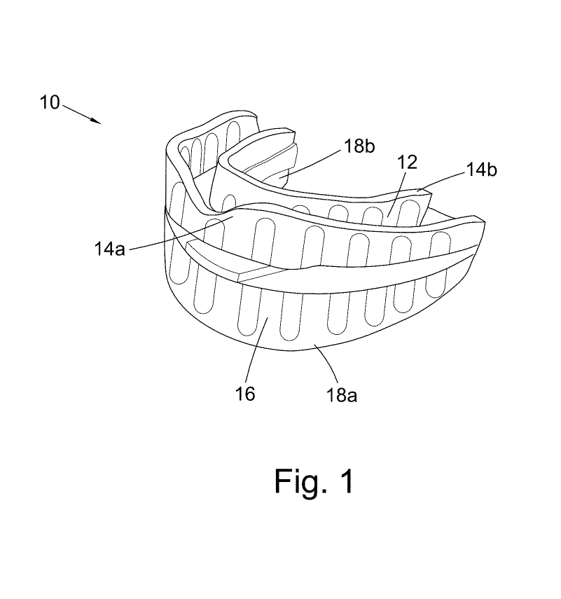

FIG. 1 illustrates a mandibular advancement device 10 as known in the prior art. Mandibular advancement device 10 is shaped to conform to the size and shape of the user's upper and lower dentures. Mandibular advancement device 10 comprises an upper groove 12 comprising a front upper wall 14a and a rear upper wall 14b. Similarly, although shown in less detail, mandibular advancement device 10 comprises a lower groove 16 comprising a front lower wall 18a and a rear lower wall 18b. Upper and lower grooves 12 and 16 are generally arc-shaped so as to follow the curvature of the user's upper and lower dentures.

As the relative positions and sizes of dentures vary from one person to the next, mandibular advancement device 10 is usually initially provided in the form of a boil-and-bite mouth guard, generally made of silicone or a similar plastic. A user first heats mandibular advancement device 10 by placing it in boiling water, so as to render it deformable. Once sufficiently heated, the user bites down in upper and lower grooves 12 and 16, thereby forming indentations in upper and lower grooves 12 and 16. Mandibular advancement device 10 rapidly cools and hardens, and in doing so mandibular advancement device 10 is moulded to the size and shape of the user's teeth, providing a relatively firm fit.

As explained above, whilst mandibular advancement devices of the type shown in FIG. 1 are useful in assisting the prevention of snoring, by causing the mandible to be advanced such that the user's airway is not blocked during sleep, such known mandibular advancement devices are none the less frequently expelled during sleep, usually from involuntary mouth movement. In addition, such mandibular advancement devices can place undue stress on the user's teeth and gums, and rely on the quality of the wearer's teeth and gums to provide at least some degree of fit in the wearer's mouth. Further, the known device of FIG. 1 requires the user's mouth to be closed, which is undesirable in an emergency situation in which airflow to the lungs is ideally maximised.

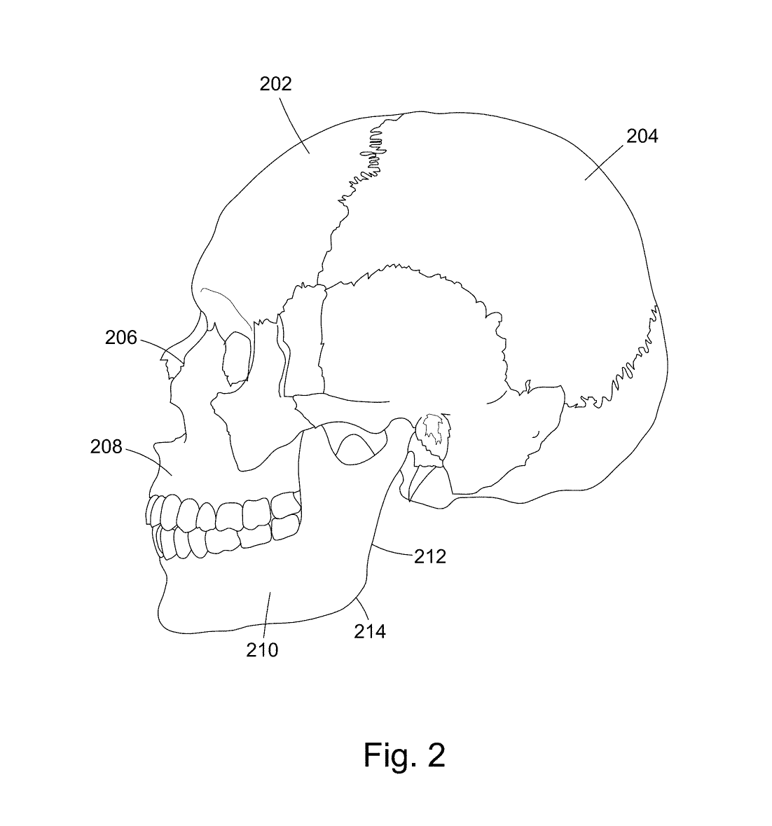

In order to aid explanation of the various embodiments described herein, FIG. 2 shows an anatomical diagram of a human skull. FIG. 2 shows an upper portion of the skull having a frontal bone 202 and a parietal bone 204. Rigidly fixed to the frontal bone 202 and the parietal bone 204 are the nasal bone 206 and the maxilla 208. The mandible of the skull, generally designated as reference 210, includes on each side a ramus 212 comprising effectively the perpendicular or outside part of the mandible, and an angle 214. The mandible 210 is able to move relative to the rest of the skull to allow opening and closing of the jaw. Particularly, the mandible 210 can move relative to the maxilla 208, the nasal bone 206, the frontal bone 202 and the parietal bone 204. The mandible 210 is sometimes termed a “lower mandible”, and the term mandible is used throughout to mean the section of the jaw that is movable relative to the maxilla 208.

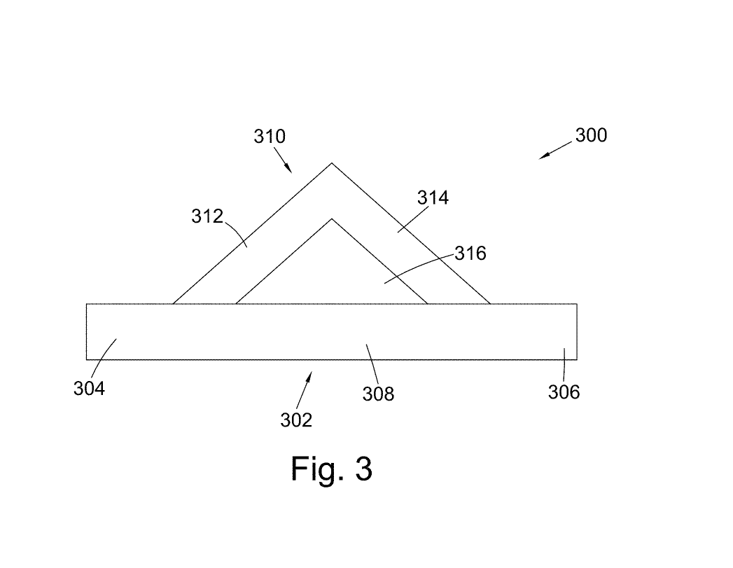

FIG. 3 shows a first embodiment of a device for fitting to a user's mandible. The device 300 includes an elongate member 302 and a support member 310. The elongate member 302 is generally formed of a rectangular strip having a first end portion 304 and a second end portion 306 located at an opposite end of the rectangular strip to the first end portion 304. A central portion 308 is located between the first end portion 304 and the second end portion 306. The central portion 308 may directly connect the first end portion 304 to the second end portion 306, or there may be further portions in between the central portion 308 and the first end portion 304, and/or in between the central portion 308 and the second end portion 306. The first end portion, central portion and second end portion (and any other portions therebetween) may be integrally formed and may be formed of the same material. Alternatively however, the first and second end portions 304 and 306 may be formed of one material, and the central portion may be formed of a different material.

The support member 310 includes a first section 312 and a second section 314. The first section 312 extends from a securement point on the elongate member 302 between the first end portion 304 and the central portion 308. In a similar manner, the second section 314 extends from a securement point on the elongate member 302 between the second end portion 306 and the central portion 308. Alternatively, the first and second sections 312 and 314 may extend from the respective first and second end portions 304 and 306. The first and second sections 312 and 314 form two sides of a triangle, with the central portion 308 forming at least part of the other side of the triangle.

In other words, the first and second sections 312 and 314 both extend from the elongate member 302 at an acute angle relative to an axis perpendicular to the length of the elongate member 302 (the length being defined as a line between the first end 304 and the second end 306). The first section 312 may therefore extend in a first direction, and the second section 314 may therefore extend in a second direction, the second direction being the reflection of the first direction in the perpendicular axis.

The first section 312 and the second section 314 meet at a point defined by an axis bisecting the elongate member 302. As such, the meeting of the first section 312 and the second section 314 define an apex of the triangle. Whilst the first section 312 and second section 314 are shown in FIG. 3 as being straight strips of material, these two sections may instead be curved such that no apex of the triangle is defined. For example, the first section 312 and second section 314 may form a single piece of material forming a curved loop connected to and extending from the elongate member 302. Indeed, any configuration of the support member 310 which defines an opening 316 between the support member 310 and the elongate member 302 may be used. For example, the first section 312 and second section 314 may have different lengths, thereby providing an asymmetric device 300.

The material of the support member 310 may be the same as the materials of the elongate member 302, or may be different therefrom. In the case where the elongate member 302 itself is formed of different materials, the support member 310 may be the same as one of the materials of the elongate member 302, or maybe different to all of the materials of the elongate member 302.

The elongate member 302 and the support member 310 may be formed of a single piece of material, and may be integrally formed. Alternatively, the support member 310 may be connected to the elongate member 302 at the points between the first end portion 304 and central portion 308, and between the central portion 308 and second end portion 306, as previously described, or the support member 310 may be connected to the first and second end portions 304 and 306. Such a connection may be by any conventional means such as stitching or adhesive.

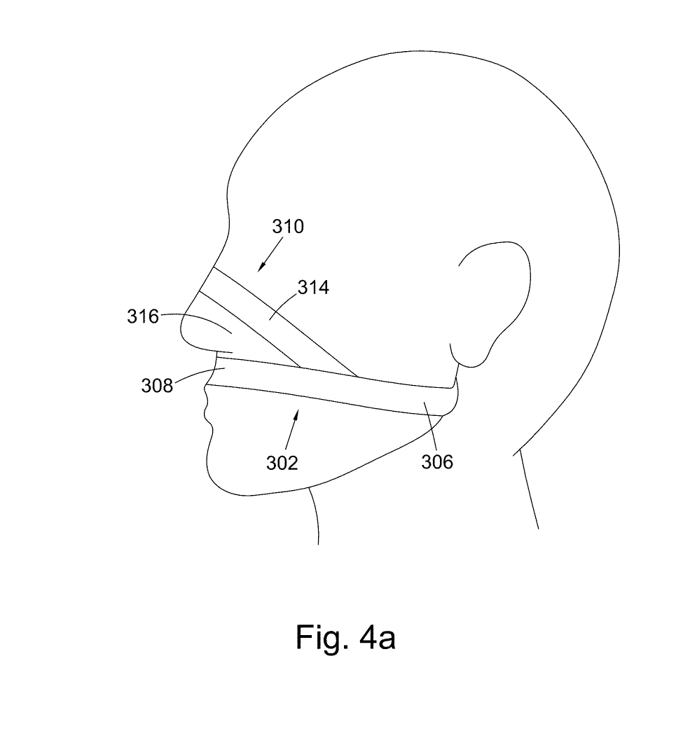

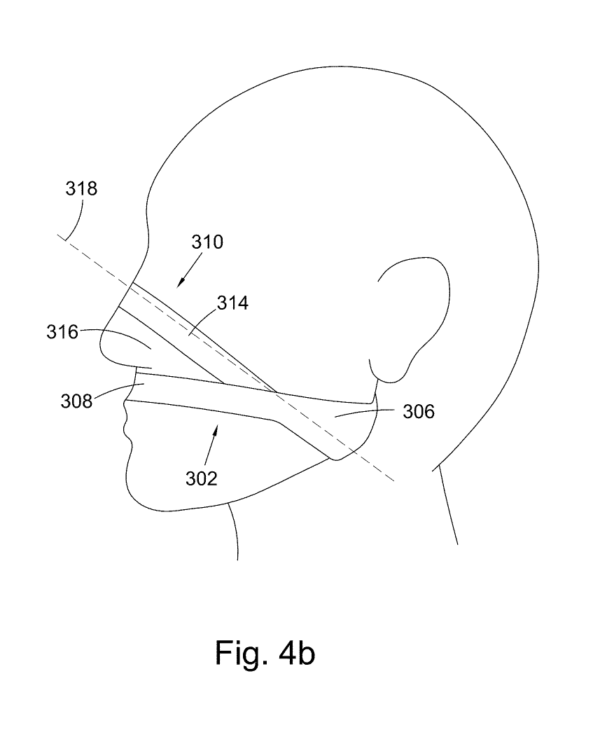

FIG. 4a shows the device 300 of the first embodiment when fitted to a user. With reference to the human skull shown in FIG. 2, the second end portion 306 of the elongate member 302 is fitted around, or is otherwise engaged with, the ramus 212 of one side of the mandible 210. The central portion 308 of the elongate member 302 wraps around and is in contact with the maxilla 208 located between the user's nose and top lip. The second section 314 of the support member 310 extends from the elongate member 302 at a point between the second end portion 306 and the central portion 308 so as to extend over and around at least part of the nasal bone 206. As can clearly be seen from FIG. 4a, the configuration of the elongate member 302 and support member 310 provides an opening 316 through which the user's nose protrudes. As would be understood, FIG. 4a simply shows one side profile of a user with the device 300 fitted thereto, and the opposite profile view would show a mirrored configuration with the first end portion 304 fitted around, or otherwise engaged with, the ramus of the mandible 210 located on the other side of the mandible 210, and the first section 312 extending between the first end portion 304 and the central portion 308. The first section 312 would also extend over and around at least part of the nasal bone 206.

By virtue of the nature and shape of the materials used for the elongate member 302, the first and second end portions 304 and 306 provide secure fittings to the two rami 212 located on opposite sides of the mandible 210. The support member 310 substantially conforms to the contours of the user's face such that the elongate member 302 lies against at least some of the user's skin. The support member 310 maintains the device 300 in a maintenance position on the user's face by preventing dislodgement of the elongate member 302 from engagement with the two rami 212. In this embodiment, the maintenance position is achieved by virtue of the support member 310 lying against at least some of the user's skin on the nasal bone 206 to prevent the elongate member 302 from sliding downwardly off the user's face. The support member 310 prevents the elongate member 302 from falling down by engaging areas of the face other than the mandible 210. The incline of the nasal bone 206 naturally angles away from the user's face, thereby providing a friction surface to resist sliding of the support member 310 down the user's nose.

The manner in which the elongate member 302 is securely fitted to the rami 212 on either side of the mandible 210 will now be described. As described in the background section, when relaxed, the mandible 210 is able to collapse and fall back towards the back of the user's head and neck. To prevent the mandible 210 from collapsing when relaxed, the elongate member 302 may be formed of a mouldable material of a length such that the first and second end portions 304 and 306 of the elongate member 302 securely engage and hook around the each ramus 212 when the mandible 210 is in the advanced or un-collapsed position. The mouldable material may be such that the user can fit the elongate member 302 across their maxilla 208 and around their rami 212 when their mandible 210 is not collapsed and, after a hardening process, the elongate member 302 material becomes rigid so as to have a fixed form in which the length of the elongate member 202 material from the ramus 212 at one side of the mandible 210 to the ramus 212 at the other side of the mandible 210 is fixed. An example material is a combination of a fibreglass substrate and a polyurethane resin system, or a non-fibreglass polyester material. Such materials are used for medical casts, such as the medical casts produced by BSN Medical®.

With the material of the elongate member 302 hardened and in this configuration, when the user relaxes their mandible 210, for example during sleep or in an emergency situation, the mandible 210 is unable to move relative to the maxilla 208 as the rigidity of the material of the elongate member 302 prevents stretching of the material and provides a tension between the first end portion 304 and the central portion 308 and the second end portion 306 and the central portion 308 to prevent collapse of the mandible 210. As the user fitted the first and second end portions 304 and 306 to their face when the material was able to be moulded, the shape of the first and second end portions, after the hardening process, will be such that the first and second end portions 304 and 306 hook around their respective ramus 212 to provide a secure fit to the user's face. The material of the device 300 may be resilient such that a slight temporary deformation of the material is possible to facilitate the fitting to a user's face.

Alternatively, the elongate member 302 may comprise an elastic material, and the first end portion 304 and the second end portion 306 may be formed of a rigid hook-shaped material. The length of the elongate member 302, including the first end portion 304 and the second end portion 306, in an un-stretched state may be shorter than the distance between one ramus 212, over the maxilla 208, and to the other ramus 212 when the mandible 210 is in the advanced or un-collapsed position. As the material of part of the elongate member 302 is elastic however, the user is able to stretch the elongate member 302 in order to hook and engage the first end portion 304 with one ramus 212 and the second end portion 306 with the other ramus 212 when the mandible 210 is in the advanced or un-collapsed position. The central portion 308 of the elongate member 302 may be the part formed of elastic material, for example.

When the elongate member 302 comprises an elastic material, the rigid material of the first end portion 304 and the second end portion 306 is shaped to securely engage with the contours of each ramus 212. As mentioned above, the first and second end portions 304 and 306 may be hook-shaped and may comprise a bent, rigid material. Such a hook-shape securely maintains the first and second end portions securely fitted to the respective ramus 212, even when a force is applied along the elongate member 302. Any shape of the first and second end portions which provides such a fitting may be used, and the shape is not limited to a hook. In other words, the first and second end portions 304 and 306 may have any shape that maintains a secure fitting between the end portions 304, 306 and their respective ramus 212 when a force is applied along the elongate member 302.

Due to the elasticity of the material, the elongate member 302 therefore urges and biases the mandible 210 into the advanced or un-collapsed position by providing a tension between the first end portion 304 and the central portion 308, and between the second end portion 306 and the central portion 308. As in the case that the elongate member 302 comprises a mouldable material, when the user relaxes their mandible 210, for example during sleep or in an emergency situation, the mandible 210 will be unable to collapse as the weight of the jaw 210 will be insufficient to stretch the elastic material of the elongate member 302. The first end portion 304 and second end portion 306 may be formed of any suitably rigid material, for example plastic.

Alternatively, the elongate member 302 may comprise a malleable material, such as a metal or metal alloy. Such a material may be easily bent by hand and maintain a desired shape. In a similar manner to the case in which the elongate member 302 is a mouldable material, the elongate member 302 can be fitted across the maxilla 208 and around the rami 212 when the mandible 210 is not collapsed. Due to the properties of the malleable material, the elongate member 302 conforms to and retains the shape of the contours of the wearer's face simply by pressing the elongate member 302 onto the wearer's face and bending the material by hand. Once the elongate member 302 is fitted, when the mandible 210 is relaxed, the mandible 210 is unable to move relative to the maxilla 208 as the weight of the maxilla 208 is insufficient to bend the malleable material out of its fitted shape. The first and second end portions 304 and 306 may be a different material, or may be integral with the other elements of the elongate member 302 and therefore also formed of the malleable material. In this case, the first and second end portions 304 and 306 may be pressed around the rami 212 by hand to hook and engage with the rami 212.

By having the elongate member 302 formed of the malleable material, the device may be quickly and easily applied to a user's face. For anti-snoring purposes, the device could be applied by the user themselves. In the case of an emergency situation however, a medical professional can quickly and easily fit the device to the user's face in order to open the user's airway, and maintain the airway open.

Similar to the elongate member 302, the support member 310 may also comprise either a mouldable, elastic or malleable material. The mouldable, elastic or malleable material may be the material of the first and/or second section, or may be a separate section between the first section 312 to the second section 314. In the case that the support member 310 comprises a mouldable material, the specific angle the first and section sections 312 and 314 make with the elongate member 302, and the contouring of the support member 310 on the user's face, may be configured by the user while the material is in a mouldable state. Then, after a hardening process, the support member 310 will be shaped such that a secure fit is provided according to the contours of the particular user's face. When hardened, the support member 310 can therefore maintain the device 300 on the user's face by preventing the elongate member 302 from sliding or falling off as described above.

In the case where the support member 310 comprises an elastic material, the un-stretched length of the support member 310 may be such that it is less than the distance on the user's face required to be covered by the support member 310, thereby requiring the elastic material to stretch. Such a stretching of the material of the support member 310 ensures that the elongate member (302) is biased and urged upwards (i.e. towards the underside of the user's nose) when the device 300 is fitted to the user's face, thereby maintaining the device 300 on the user's face.

In the case where the support member 310 comprises a malleable material, the contouring of the support member 310 on the user's face, and optionally the specific angle the first and section sections 312 and 314 make with the elongate member 302, may be configured by the user or another person by pressing the support member 310 against the user's face. The malleability of the material is such that the device conforms to the contours of the user's face to provide a good fit and prevent the device 300 from easily sliding off the user's face.

Regardless of whether the support member 310 comprises a mouldable, elastic or malleable material, the result is that the support member 310 is maintained and held on the user's face by virtue of the engagement of the first section 312 and the second section 314 with the user's face.

FIG. 4b shows a second embodiment of the device 300. The first end portion 304 and the second end portion 306 are shown in FIG. 3 as simply being the square ends of rectangular strip elongate member 302. However, each of the first and portion 304 and the second end portion 306 may take a different shape. In the second embodiment shown in FIG. 4b for example, each end portion is instead shaped to “fan” out such that the elongate member 302 is wider at the first and second end portions than in the central portion 308. The other features of the second embodiment are the same as the first embodiment shown in FIG. 4a. As is clear from FIG. 4b, since the end portions fan out (shown in FIG. 4b by the shape of the second end portion 306), each end portion is able to engage not only the contours of the ramus 212, but also the angle 214 of the mandible. This increases the security of the fitting of the device 300 on a user's face.

First, the end portions can now engage two separate locations of the user's mandible, the ramus and the angle, thereby increasing the contact surface area. Second, the position of each angle 214 is such that there is a line of tension from the part of the end portion that contacts the angle 214, along the respective first section 312 or second section 314, and to the bridge of the nose. This is possible by having a part of each end portion that is aligned with the length of the respective first section 312 or second section 314. This is shown in FIG. 4b as a part of the second end portion 306 being aligned with the length of the second section 314. As a result, a direct line of tension 318 is provided from the part of the second end portion 306 that engages the angle 214, along the second section 314, and over the bridge of the nose. Due to this line of tension 318, the support member 310 can be sized and/or shaped to provide a tighter fit without compromising comfort since the support member 310 directly pulls against parts of the first and second end portions that engage the respective angles 214.

Alternatively, each of the first end portion 304 and the second end portion 306 may be divided into two or more separate end portions. In other words, the elongate member 302 may be a single strip at the central portion 308, and may split along its length as it approaches the end portions, thereby forming multiple end portions at each of the first end portion 304 and the second end portion 306. For example, the second end portion 306 may comprise two end portions, one end portion engaging the ramus 212 and another end portion engaging the angle 214. As above, the end portion engaging the angle 214 may be aligned with the length of the respective first or second section, to provide the line of tension 318. Therefore, this alternative also provides a tighter fit without compromising comfort, and increases the contact surface area.

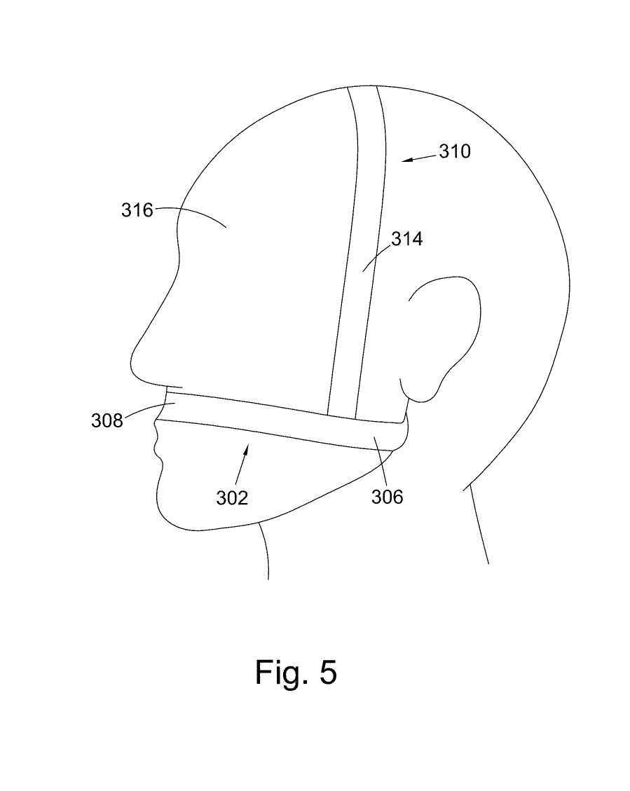

FIG. 5 shows a third embodiment of the device 300. The details of this third embodiment are the same as those given for the first embodiment; however the support member 310 has an increased length such that the support member 310 can extend over the top of the user's head while in use. This may be achieved by increasing the length of the first and/or second section, and/or by increasing the length of any mouldable, elastic or malleable material between the first and second sections. In other words, the first section 312 and the second section 314 extend substantially perpendicular to the length of the elongate member 302, so as to form a loop or part of a loop that encircles the top of the user's head. As for the first embodiment, the first section 312 may be attached between the first end portion 304 of the elongate member 308 and the central portion 308, and the second section 314 may extend from a point between the second end portion 306 of the elongate member 302 and the central portion 308. Alternatively, the first and second sections 312 and 314 may extend from the respective first and second end portions 304 and 306.

The support member 310 of the third embodiment may also comprise a mouldable, elastic or malleable material, as described in the first embodiment. In a similar manner to that described in relation to the first embodiment, in the case that the support member comprises a mouldable material, the specific angle that the first and section sections 312 and 314 make with the elongate member 302, and the contouring of the support member 310 over the top of the user's head, may be configured by the user while the material is in a mouldable state. Then, after a hardening process, the support member 310 will be shaped such that a secure fit is provided across the top of the user's head. When hardened, the support member 310 can therefore maintain the device 300 on the user's face by preventing the elongate member 302 from sliding or falling off due to the support member 310 passing over the top of the user's head.

In a similar manner to that described in relation to the first embodiment, in the case that the support member 310 of the third embodiment comprises an elastic material, the length of the support member 310 may be such that a section of the support member 310 must be stretched in order for the support member 310 to extend over the user's head. The section that is made of the elastic material may be the first section 312, the second section 314, or a separate section between the first section 312 and the second section 314. In such a configuration, the support member 310 exerts a tension along the first and second sections 312 and 314 in order to bias the elongate member 302 upwards generally towards the top of the user's head. Depending on the point to which the first and second sections 312 and 314 are joined to, or which they are integral with, the elongate member 302, the bias provided by the first and second sections may also act to urge the first and second end portions 304 and 306 upwards towards the user's ear. In the case that the first and second sections 312 and 314 extend from the respective first and second end portions 304 and 306, substantially the entirety of the bias may be upwards towards the user's ears.

In the case that the support member 310 comprises an elastic material, depending on the elasticity of the material and the size of the user's head, it may be possible for the device of the first embodiment shown in FIG. 4a and the second embodiment shown in FIG. 4b to be fitted to the user in the same manner as that shown in the third embodiment of FIG. 5. In other words, it may be possible for the support member 310 shown in FIG. 4a or FIG. 4b to be stretched and fitted to a user as shown in FIG. 5.

In a similar manner to that described in relation to the first embodiment, in the case that the support member 310 comprises a malleable material, the contouring of the support member 310 over the top of the user's head, and optionally the specific angle that the first and section sections 312 and 314 make with the elongate member 302, may be configured by the user or another person by pressing the support member 310 against the user's head.

Although in the third embodiment the support member 310 is shown as extending over substantially the highest point of the user's head, the support member 310 may extend over any area defined by the parietal bone or indeed may also or alternatively extend over the frontal bone. The angle between the first and second sections 312 and 314 of the support member 310 and the length of the elongate member 302 will be defined by the particular configuration in which the device is fitted to the user's head.

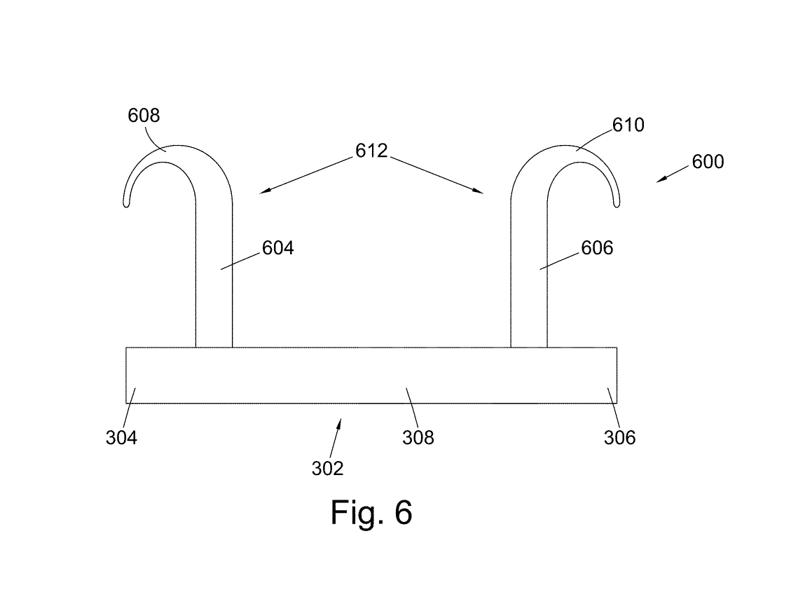

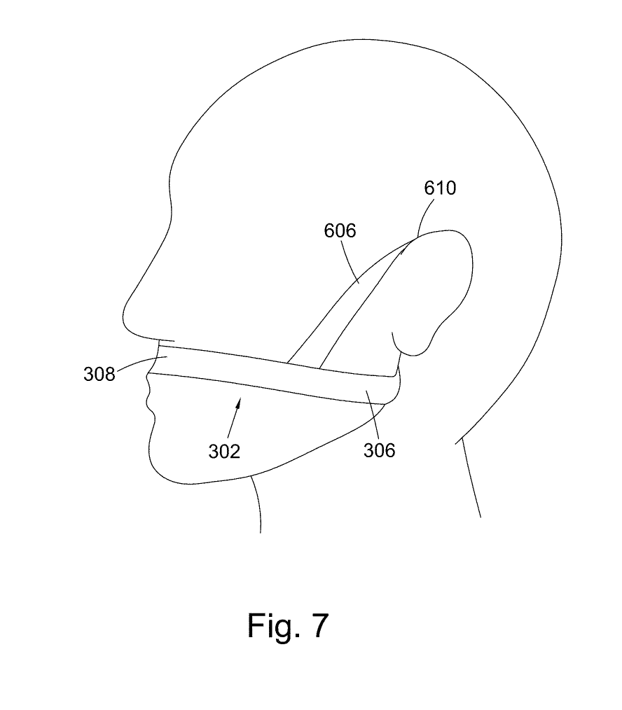

FIGS. 6 and 7 show a fourth embodiment of the device. The device 600 shown in FIGS. 6 and 7 includes the elongate member 302 as described in the previous embodiments. The various features of the elongate member 302 in this fourth embodiment are therefore the same as in the previous embodiments. The device 600 however does not have the support member 310, and instead has two support members generally designated as 612 in FIG. 6. One of the support members 612 has a first section 604 extending from the elongate member 302 at a point between the first end portion 304 and the central portion 308, or from the first end portion 304. At an end of the first section 604 opposite to the point of connection to the elongate member 302, there is a first hook 608 formed of a curved structure. The device 600 also has a second support member 612 formed of a second section 606 extending from the elongate member 302 at a point between the second end portion 306 and the central portion 308, or from the second end portion 306. At an end of the second section 606 opposite the point of connection to the elongate member 302, there is a second hook 610 also formed of a curved structure.

The first hook 608 and second hook 610 are such that they can pass around each ear of the user between the helix of the each ear and the respective side of the user's head.

The first hook 608 and second hook 610 therefore go around the user's ears in the same manner as that achieved by conventional glasses/spectacles. The first hook 608 and the second hook 610 may comprise a mouldable material that can be shaped and hardened (such as the mouldable materials previously described in relation to the previous embodiments), a malleable material (such as the malleable material previously described in relation to the previous embodiments) or may comprises a rigid material having a fixed curved structure.

Again, similar to the mouldable, elastic and malleable materials described in relation to the previous embodiments, the first section 604 and second section 606, and/or a different section of the support members 612, may also comprise any one of these materials. In the case that the first section 604 and the second section 606 comprise mouldable materials, the first section 604 and second section 606 may be shaped by a user to lie along the user's face between their ear and their nose, generally across their cheek. Then, after the hardening process, the first section 604 and second section 606 securely lie against the contours of the particular user's face to maintain the device 600 on the user's face, as shown in FIG. 7.

As such, while the first section 604 and the second section 606 are in a mouldable state, the user may ensure that the respective first hook 608 and second hook 610 fits securely around their ears to prevent the elongate member 302 from sliding or falling off the user's face.

In the case that the first section 604 and the second section 606 comprise an elastic material, and/or another section of the support members 612 comprises an elastic material, the length of the first section 604 and second section 606, and/or the other section, may be such that one or more of these sections need to stretched in order that the hooks 608 and 610 can pass around a user's ear (between the helix and the side of the user's head) to exert a tension along the respective first and second sections 604 and 608. This configuration therefore also biases the elongate member 302 generally towards the top of the user's head, and specifically towards the underside of the user's nose and/or towards the user's ear, to maintain the device 600 on the user's face. In this configuration, the hooks 608 and 610 will either be pre-moulded and hardened by a user prior to fitting the device 600 to the user's face, or the hooks will be formed of a rigid material or a malleable material.

In the case that the first section 604 and the second section 606 comprise a malleable material, the first section 604 and second section 606 may be shaped by a user or another person to lie along the user's face between their ear and their nose, generally across their cheek. Such a shaping may be created by pressing the material against the user's face such that the first section 604 and second section 606 securely lie against the contours of the particular user's face, thereby maintaining the device 600 on the user's face, as shown in FIG. 7.

While FIG. 7 only shows one side profile view, the skilled person would readily understand that, as is clear from FIG. 6, the device 600 has a mirrored configuration when looking at the other side of the user's head as well.

An advantage of the fourth embodiment is that the support members 612 do not need to pass around the user's head or face, and instead can be worn in a manner similar to conventional glasses/spectacles which may be more comfortable and which the user may already be used to. The user may therefore find that the configuration of the third embodiment has increased comfort and is less disruptive to their sleep.

As for the embodiment of FIG. 4b, any of the embodiments of FIGS. 5-7 may also have end portions that “fan” out. Such end portions allow engagement with both the ramus and the angle on each side of the mandible, thereby increasing the contact surface area as described above in relation to FIG. 4b.

In a fifth embodiment of the device, the fourth embodiment is modified such that each support member 612 is retained by the user's ear in a manner similar to ear phones. In other words, the hooks 608 and 610 are instead each shaped to be retained in an inferior part of the earlobe to provide a secure fitting to the ear. Alternatively or additionally, the hooks 608 and 610 may each have a portion shaped to fit inside the ear to provide a secure fitting to the ear. Indeed, the hooks may be shaped to fit to any internal portion of the ear. The hooks 608 and 610 of the fifth embodiment therefore provide an alternative manner of securing the device to a user.

As is clear from the embodiments described above, the invention disclosed herein provides an anti-snoring device or an emergency care device which is securely fitted to a user's face, without requiring insertion of any objects into the user's mouth or monitoring by another person. The device is prevented from falling off the user's face by virtue of the support member that either rigidly fixes the device to match the contours or shape of a user's face, and/or provides a bias force generally towards the top of the user's head. Additionally, as there is no reliance on adherence to the teeth or gums, users with poor tooth or gum health, or indeed user's with no teeth at all, may easily wear the device to prevent snoring. The device may therefore be particularly suitable for older users who generally have worsened oral conditions. Further, the secure hold provided by the device ensures that the user's airway is opened and remains open in an emergency situation.

As the device may comprise mouldable or malleable materials, the device can be specifically tailored to the contours and shapes of each individual user's face and head, thereby ensuring a secure fit that is able to maintain the mandible in an advanced or un-collapsed position. Such an advanced or un-collapsed position may therefore be maintained at all times without requiring monitoring or other assistance, for example during sleep or in an emergency situation in which the attention of a medical professional is directed elsewhere. By creating and maintaining the uncollapsed or advanced position of the mandible, the user's airway is opened and maintained open in a quick and efficient manner.

If elastic materials are used, the device may be quickly and easily fitted to the user and provide a “one size fits all” device.

Whilst the invention has been described in connection with preferred embodiments, it would be understood that the invention is not limited to these embodiments, and that alterations, modifications, and variations of these embodiments may be carried out by the skilled person without departing from the scope of the invention. For example, whilst the invention has been described primarily in the context of an anti-snoring device or an emergency care device, its use and applicability may extend more generally to other devices in which it is beneficial to quickly and easily fit a device to a user's face or head to advance the mandible and open the airway.

1. A device for fitting to a user's mandible such that the mandible is held in an advanced position, the device comprising:
an elongate member comprising a first end portion, a central portion, a second end portion and at least one securement point; and
a support member extending from the securement point to, in use, maintain the device in a maintenance position on the user's face;
wherein the first end portion is arranged to engage one ramus of the mandible, the second end portion is arranged to engage the other ramus of the mandible, the central portion is arranged to engage a first area of the user's face or head other than the mandible, and the support member is arranged to engage a second area of the user's face or head other than the mandible.

2. The device of claim 1, wherein the support member comprises a first section attached to a first securement point of the elongate member between the first end portion and the central portion, and a second section attached to a second securement point of the elongate member between the second end portion and the central portion.

3. The device of claim 2, wherein the first and second sections together form a loop arranged such that, in use, the user's nose protrudes between the elongate member and the support member.

4. The device of claim 1, wherein the first end portion is further arranged to engage one angle of the mandible, and the second end portion is further arranged to engage the other angle of the mandible.

5. The device of claim 2, wherein the first section has a first length and the second section has a second length, and wherein each end portion comprises an angle engagement portion arranged to engage an angle of the mandible, each angle engagement portion being aligned with the respective first or second length.

6. The device of claim 1, wherein the first area comprises the user's maxilla or nasal bone.

7. The device of claim 1, wherein the second area comprises the user's nasal bone, the user's frontal bone or the user's parietal bone.

8. The device of claim 2, wherein the first section is arranged to engage one of the user's ears, and the second section is arranged to engage another of the user's ears.

9. The device of claim 8, wherein the first section is arranged to engage one of the user's ears behind the helix of the ear, and the second section is arranged to engage the other of the user's ears behind the helix of the other ear.

10. The device of claim 1, wherein at least part of the elongate member or the support member comprises a mouldable material.

11. The device of claim 10, wherein the mouldable material of the elongate member is arranged to be moulded such that the engagement of the first and second end portions with the two mandibular rami, and the engagement of the central portion with the first area, is provided.

12. The device of claim 10, wherein the mouldable material of the support member is arranged to be moulded such that the engagement with the second portion is provided.

13. The device of claim 1, wherein at least part of the elongate member or the support member comprises an elastic material.

14. The device of claim 1, wherein first and second end portions are further arranged to engage the mandibular angle adjacent to each ramus.

15. The device of claim 1, wherein the elongate member is arranged such that, when the first and second end portions are engaged with the two mandibular rami, and the central portion is engaged with the first area, a bias force is exerted between the first end portion and the central portion and between the second end portion and the central portion, the bias force being such that the user's mandible is biased into the advanced position, the bias force being in a direction substantially along a line passing from the first or second end portion to the central portion.

16. The device of claim 1, wherein the first and second end portions are mouldable to engage the respective first and second mandibular ramus.

17. The device of claim 1, wherein at least one of the first end portion, the second end portion, the central portion, and the support member is stretchable and/or resilient to provide the fitting to a user's mandible.

18. The device of claim 1, wherein the elongate member and/or the support member comprise a malleable material.

19. A device for fitting to a user's mandible such that the mandible is held in an advanced position, the device comprising:
an elongate member comprising a first end portion, a central portion, a second end portion and at least one securement point; and
a support member extending from the securement point to maintain the device in a maintenance position on the user's face;
wherein the first end portion engages one ramus of the mandible, the second end portion engages the other ramus of the mandible, the central portion engages a first area of the user's face or head other than the mandible, and the support member engages a second area of the user's face or head other than the mandible.

20. A method of fitting the device of claim 1 to a user's mandible such that the mandible is held in an advanced position, the method comprising:
while at least part of the elongate member is mouldable and while the mandible is in the advanced position, placing the elongate member across the user's face such that the first and second end portions extend around the each mandibular ramus, and the central portion is in contact with the first area of the user's face or head;
placing the support member over the second area of the user's face or head;
bending the first end portion around one ramus to engage the first end portion with the ramus;
bending the second end portion around the other ramus to engage the second end portion with the other ramus;
waiting a period of time or providing a hardening process, such that the mouldable material of the elongate member becomes rigid.
